(19) United States
(12) Patent Application Publication
(10) Pub. No.: US 2023/0321301 A1
(43) Pub. Date: Oct. 12, 2023

(54) VOLATILE DISPENSING DEVICE WITH DISPENSER MOUNT

(71) Applicant: The Procter & Gamble Company, Cincinnati, OH (US)

(72) Inventors: Rahul VYAS, Singapore (SG); Desmond NG, Singapore (SG); Mei San Gigi CHO, Singapore (SG); Maggie SEAH, Singapore (SG); Bhavesh RAJWANI, Singapore (SG); William Michael CANNON, West Harrison, IN (US)

(21) Appl. No.: 18/192,705

(22) Filed: Mar. 30, 2023

Related U.S. Application Data
(60) Provisional application No. 63/329,486, filed on Apr. 11, 2022.

(51) Int. Cl. A61L 9/04; A61L 9/12

(52) U.S. Cl. CPC A61L 9/042; A61L 9/12; A61L 2209/133; A61L 2209/15

(57) ABSTRACT

A volatile dispensing device having a housing having opposing first and second walls that are joined along their peripheries to one another. Each of the first and second walls has at least one aperture and define a cavity for receiving a solid article having a volatile composition. A dispenser mount is located at a center of one of the first wall and the second wall and extending from an exterior surface of the one of the first wall and the second wall, wherein the dispenser mount has a planar mount surface and is characterized by a height greater than a thickness of the one of the first wall and the second wall.

### FIELD OF THE INVENTION

The present invention relates to volatile composition dispensers for delivering a volatile composition, and particularly relates to a volatile dispensing device with a dispenser mount, and a volatile composition dispenser with a dispenser mount.

### BACKGROUND OF THE INVENTION

The use of various devices for diffusion of volatile compounds such as perfumes, sanitizing agents, insect repellants, air freshening compositions, deodorizers are well known. Passive volatile composition dispensers are a popular means for delivering volatile compounds into the air. A passive volatile composition dispenser may include a container containing a volatile composition that is delivered to the surrounding atmosphere through vents in the container. The dispenser may be designed to be used in a free-standing mode where there is a suitably flat surface to place it. The dispenser may also be designed to be used in a hanging mode where there is a hook for hanging the device to a support.

In particular, a dispenser which utilizes a solid carrier for delivering a volatile compound typically have a surface for receiving the solid carrier. A gel air freshener device with a gel receiving surface having a recess for receiving a gel composition is described in UK Patent Application Publication No. GB2374805A. However, only a top side of the gel composition (not in contact with the gel receiving surface) exposed to the atmosphere thereby limiting the delivery of volatiles in the gel composition to one side. Further, deployment of the device is also limited as the device needs to be positioned such that the gel composition does not come into contact with an interior surface of placement of the device. Contact of volatile compounds in the gel composition with the interior surface may result in unintended alteration of the interior surface especially if the volatile compounds are incompatible with surface finishes on the interior surface such as for example paint finishing.

U.S. Pat. No. 9,591,842B2 describes a passive volatile dispensing device including a housing having first and second curved walls and sidewalls between the curved walls. A volatile-bearing substrate is disposed within an internal cavity of the housing. A plurality of apertures are provided on both of the first and second walls to release an active from the volatile-bearing substrate. The volatile-bearing substrate is a cellulosic substrate that is impregnated with and/or coated with an active ingredient for diffusion into the surrounding atmosphere. However, the substrate may sit too far into the apertures of the walls and as such, the device requires features such as rib design in the housing to secure the substrate within the housing so as to prevent unintended contact by the substrate with an interior surface of placement of the device. The addition of features creates complexity in manufacturing and assembly.

In view of the above, there remains a need for a volatile composition dispenser that can be used to contain a solid article comprising a volatile composition and can be used in a variety of locations such as being attached to a door, a wall or interior furnishings or placed in a free-standing manner in an interior space to deliver a volatile composition while preventing unintended contact of the solid article with surfaces in the interior space.

### SUMMARY OF THE INVENTION

The present invention relates to a volatile dispensing device comprising:
a) a housing having opposing first and second walls that are joined along their peripheries to one another, wherein each of the first and second walls has at least one aperture and define a cavity for receiving a solid article comprising a volatile composition;
b) a dispenser mount located at a center of one of the first wall and the second wall and extending from an exterior surface of the one of the first wall and the second wall, wherein the dispenser mount comprises a planar mount surface and is characterized by a height greater than a thickness of the one of the first wall and the second wall.

### BRIEF DESCRIPTION OF DRAWINGS

FIG. 1 is a front perspective view of a volatile dispensing device according to the present invention;

FIG. 2 is a rear perspective view of the volatile dispensing device of FIG. 1;

FIG. 3 is a side view of the volatile dispensing device of FIG. 1;

FIG. 4 is a rear view of the volatile dispensing device of FIG. 1;

FIG. 5 is a rear view of an alternative embodiment of a volatile dispensing device according to the present invention;

FIG. 6 is a perspective view of components in a volatile composition dispenser according to the present invention;

FIG. 7 is a side schematic view of the assembled volatile composition dispenser of FIG. 6 in a vertical orientation when the dispenser is placed/attached to an interior surface or support; and

FIG. 8 is a side schematic view of the assembled volatile composition dispenser of FIG. 6 in a horizontal position when the dispenser is placed/attached to an interior surface or support.

### DETAILED DESCRIPTION OF THE INVENTION

It has been found that versatility in placement of consumer products in a variety of locations play an important role in enabling consumers/users to use consumer products which contain volatile compositions customized for delivering a benefit in an interior space at a specific location. In particular, it may be preferred to separate solid articles comprising volatile compositions from direct contact with interior spaces through design of a volatile composition dispenser having a container with an aperture-free base for receiving the solid articles to minimize any risk of incompatibility or adverse reactions of the solid article material and/or volatile compositions with the interior surfaces on which the container is placed. However, as a result, the volatile compositions may become trapped within the container resulting in poor performance of the volatile composition dispenser.

The present invention relates to a volatile dispensing device comprising a housing having opposing first and second walls that are joined along their peripheries to one another, wherein each of the first and second walls has at least one aperture and define a cavity for receiving a solid article comprising a volatile composition. A dispenser mount is located at a center of one of the first wall and the second wall, wherein the dispenser mount extends from an exterior surface of the one of the first wall and the second wall. The dispenser mount comprises a planar mount surface and is characterized by a height from the planar mount surface to the center of the one of the first wall and the second wall, wherein the height is greater than a thickness of the one of the first wall and the second wall.

A technical effect of the dispenser mount having a planar mount surface and a height greater than a thickness of the one of the first wall and the second wall is that the exterior surface of the one of the first wall and the second wall can be spaced away from the interior surface on which the volatile dispensing device is placed. Advantageously, in use when the solid article is within the cavity defined by the first and second walls, the solid article may be spaced apart from the interior surface thereby preventing and/or minimizing contact between the solid article and the interior surface.

In the following description, the dispenser described is a consumer product, such as an air freshener, for evaporating a volatile composition in spaces to deliver a variety of benefits such as freshening, malodor removal or scenting of air in spaces such as rooms in household and commercial establishments, or enclosed spaces such as a vehicle passenger compartment space. However, it is contemplated that the dispenser may be configured for use in a variety of applications to deliver a volatile composition to the atmosphere and the dispenser may include but is not limited to consumer products, such as, for example air freshening products.

Prior to describing the present invention in detail, the following terms are defined for clarity. Terms not defined should be given their ordinary meaning as understood by a skilled person in the relevant art.

“Interior space” as used herein refers to a finite volume of space in a residential, commercial, vehicle environment or an enclosed space. The interior space may include for example a sanitary facility such as a bathroom, a toilet, a bathroom containing a toilet, a locker room. The enclosed space may include such as for example, furniture for storage of personal items including but not limited to shoe cabinets, wardrobes, gym lockers, as well as trash cans, waste collection bins, pet litter boxes, cat litter, pet cages, pet carriers. Pet carriers are small portable boxes, crates, or cages used to transport small animals such as cats, lap dogs, hamsters, guinea pigs or the like, from one location to another.

“Horizontal orientation” as used herein, refers to a position of a volatile composition dispenser according to the present invention wherein a central evaporative surface is facing the environment in an upward or downward position.

“Solid article” as used herein, refers to a three-dimensional object having a width, length and thickness along an x-axis, y-axis, and z-axis, respectively. The solid article is self-supporting and comprises at least two evaporative surfaces.

“Vertical orientation” as used herein, refers to a position of a volatile composition dispenser according to the present invention wherein a central evaporative surface is facing the environment in a forward-facing position or in a rear facing position.

“Non-energized” as used herein, means that the volatile composition dispenser is passive and does not require to be powered by a source of external energy. In particular, the volatile composition dispenser does not need to be powered by a source of heat, gas, or electrical current, and the volatile composition is not delivered by aerosol means. Further, as used in this specification and the appended claims, the singular forms “a”, “an”, and “the” include plural references unless the content clearly dictates otherwise.

“Top notes” as used herein, refer to perfume raw materials having a high volatility.

“Bottom notes” as used herein, refer to perfume raw materials which are less volatile relative to the top notes.

“Vapor” as used herein, refers to a gaseous form of an organic or inorganic substance which coexists as a solid or liquid at ambient conditions including but not limited to temperature, humidity, and air pressure.

“Vapor impermeable substrate” as used herein, refers to a material configured to resist diffusion of vapor from the dispenser prior to its intended use.

“Vapor release rate” as used herein, refers to a measure of the passage of vapor through a substrate.

“Volatile composition” as used herein, refers to a material that is vaporizable at room temperature and atmospheric pressure without the need of an additional energy source. The volatile composition may be configured for various uses, including but not limited to, air freshening, deodorization, odor elimination, malodor counteraction, pest control, insect control, insect repelling, medicines/medicaments, disinfectants, sanitization, mood enhancement, aromatherapy aid, scented compositions, non-scented compositions, or any other use which requires a volatile composition that acts to condition, modify, or otherwise change the atmosphere or the environment. Further, it is not necessary for all of the component materials of the volatile composition to be volatile. Any suitable volatile composition in any amount or form, including a liquid, solid, gel or emulsion, may be used. Materials suitable for use herein may include non-volatile compounds, such as carrier materials (e.g., water, solvents, etc.). It should also be understood that when the volatile composition is described herein as being “delivered”, “emitted”, or “released”, this refers to the volatization of the volatile component thereof and does not require that the non-volatile components thereof be emitted.

For the purposes of illustrating the present invention in detail, the invention is described below as a non-energized volatile composition dispenser. However, the volatile composition dispenser may be configured for use with an energized device such as, for example, an electrical heating device or a fan. The solid article described hereinafter is a gel composition comprising a perfume as an example of a volatile composition. The gel composition is polyester polyol cross-linked using a cross-linking agent selected from the group consisting of: isocyanates, isothiocynates and mixtures thereof. However, it will be appreciated that the solid article may be formed from any gel composition that can be molded into three-dimensional objects and that when set, is self-supporting and has at least a peripheral evaporative surface and a central evaporative surface. Further the solid article may also be a permeable substrate that is impregnated with and/or coated with the volatile composition. The permeable substrate may be a cellulosic substrate.

The volatile dispensing device and the solid article may be configured in a variety of shapes and sizes to facilitate customization for use as volatile composition dispensers such as air fresheners in vehicles, residential interior spaces, commercial interior spaces, a household furniture interior space such as cupboards or lockers, a household appliance interior space. Preferably the household appliance may be selected from the group consisting of: refrigerators, air conditioners, washing machine, automatic dishwashing machine. The interior space may be a portable consumer product interior environment, preferably the portable consumer product may be bags, luggage or the like.

A solid article comprising a volatile composition for delivering a benefit can be implemented using a volatile dispensing device, such as an apparatus for delivering a volatile composition into an interior space. It is contemplated that the apparatus may be configured for use in a variety of applications to deliver volatile materials to the atmosphere and/or a surface as long as the volatile material is evaporated from the apparatus. For the purposes of this disclosure, but without intending to limit the scope of the invention, the apparatus is described as a non-energized apparatus.

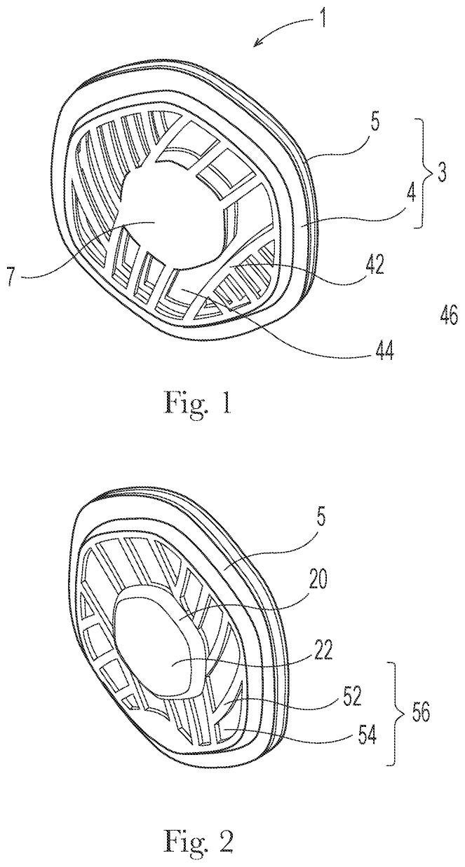

FIG. 1 is a front perspective view of a volatile dispensing device 1 according to the present invention. FIG. 2 is a rear perspective view of the volatile dispensing device 1. The volatile dispensing device 1 comprises a housing 3 having a first wall 4 and a second wall 5 opposite to the first wall 4. Each of the first and second walls 4, 5 has at least one aperture and can be releasably attached and/or joined together to define a cavity 6 for receiving a solid article comprising a volatile composition. The first wall 4 may define a front cover for the housing 3. The first wall 4 may comprise a central portion 7 having a front surface for applying a decorative element thereon. The decorative element may be a logo or a graphical symbol. The first wall 4 may further comprise a plurality of first wall solid portions 42 and a plurality of first wall apertures 44 which are interconnected to define a first peripheral wall region 46 surrounding the central portion 7.

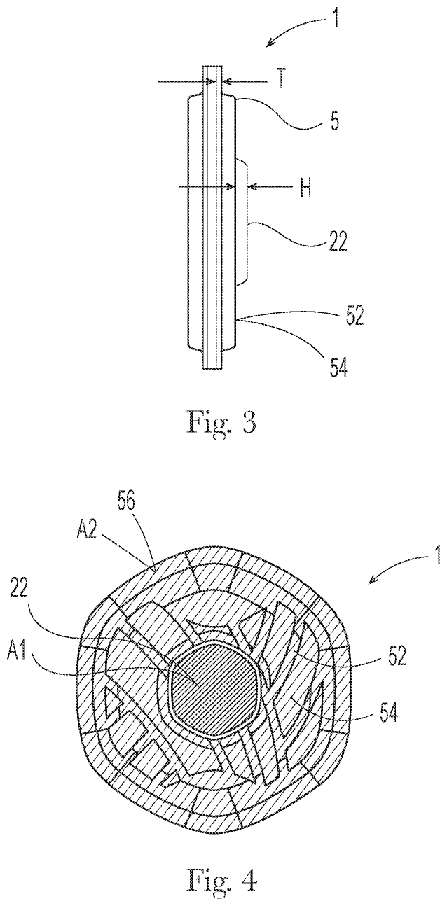

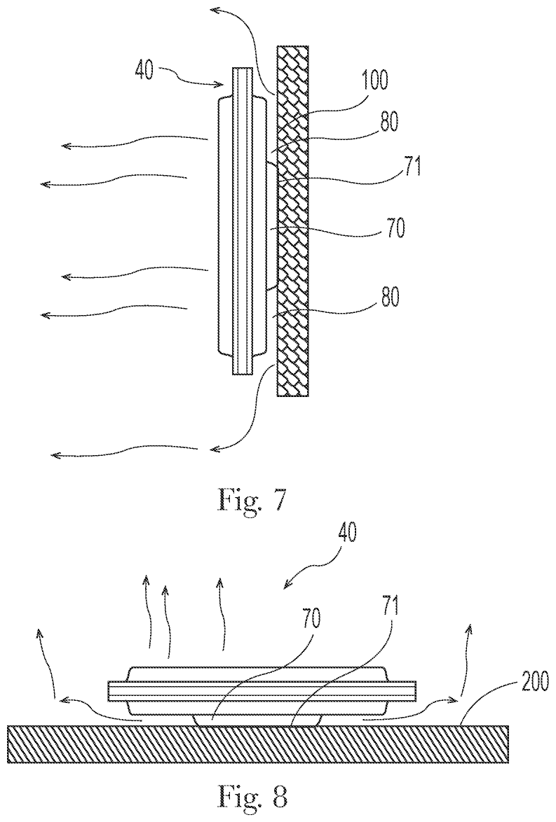

The second wall 5 may define a rear frame for the housing 3. Referring to FIG. 2, the second wall 5 comprises a dispenser mount 20 located at a center of the second wall 5. The dispenser mount 20 comprises a planar mount surface 22 that is substantially flat for supporting and/or attaching the device 1 in a vertical orientation (as shown in FIG. 7) or in a horizontal orientation (as shown in FIG. 8) to an interior surface or support in an interior space. The dispenser mount 20 is characterized by a height H greater than a thickness T of the second wall 5 as shown in FIG. 3 so as to provide a gap between the device 1 and the interior surface thereby preventing direct contact of a solid article with the interior surface.

Referring to FIG. 2, the second wall 5 may comprise a plurality of second wall solid portions 52 and a plurality of second wall apertures 54 which are interconnected to define a second peripheral wall region 56 surround the dispenser mount 5. The height H of the dispenser mount 5 may be determined by measuring a height between the planar mount surface 22 to the exterior surface of at least one of the second wall solid portions 52. As shown in FIG. 2, the dispenser mount 20 is integral with the second wall 5. However, it will be appreciated that that the dispenser mount 20 may be integral with the first wall 4 depending on an application of the device 1 in an interior space.

The constituents of the housing 3, including the first and second walls may be made from plastic, bamboo, wood, glass, shell, pulp, metals, or metalloids. It is also foreseeable in certain embodiments that the selected material of the walls may be recyclable or even made from recyclable materials. Any of the components of the housing which include the first and second walls as well as the dispenser mount 20 may be molded via thermal means, injection, or by blowing. Specifically, at least one of the first and second walls may comprise a material selected from the group consisting of: molded pulp paper, plastic, glass, ceramic and a mixture thereof.

Referring to FIG. 3, these first and second walls 4, 5 are joined to each other along their respective peripheries or through an article holder 60 disposed between the first wall 4 and the second wall 5. The article holder 60 may configured for holding a solid article in a predetermined position within the housing (which will be described hereinafter with reference to FIG. 6). The first and second walls 4, 5 may be hingedly connected to form a unitary housing piece. The dispenser mount 20 may be characterized by a thickness corresponding to a thickness of the second wall 5 for ease of manufacturing of the housing 3 as a unitary housing piece.

These walls may be joined to one another by various mechanisms including snap fit connectors, glue, or one or more latches that mechanically attaches one of the walls to the other. The first wall 4 and second wall 5 may be individually planar, convex and may even be so convex as to form two hemispherical walls separately and a sphere-shaped dispenser when joined to one another. In the depicted embodiment, however, the first wall 4 and second wall 5 are each having a substantially planar exterior surface in a hexagonal shell form. Therefore, in this instance, they form a hexagonal shaped disc housing and dispenser. However, it will be appreciated that the housing 3 may be formed in any shape based on a size and shape of the solid article or solid articles disposed therein.

FIG. 4 is a rear schematic view of the device 1 of FIG. 1 which shows a detailed view of the second wall 5. The planar mount surface 22 is characterized by a mount surface area A1 and the second peripheral wall region 56 is characterized by a peripheral wall region area A2. The ratio of the mount surface area A1 to the second peripheral wall region area A2 may be from 1:2 to 1:15, preferably from 1:5 to 1:15, more preferably from 1:7 to 1:10. The mount surface area A1 may be from 100 mm² to 400 mm², from 150 mm² to 350 mm², from 200 mm² to 300 mm² or different combinations of the upper and lower values described above or combinations of any integer in the ranges listed above. The second peripheral wall region area A2 may be from 1000 mm² to 4000 mm², from 1200 mm² to 3800 mm², from 1500 mm² to 3500 mm², from 1800 mm² to 300 mm², or different combinations of the upper and lower values described above or combinations of any integer in the ranges listed above.

The second peripheral wall region area A2 includes the respective area of the second wall solid portions 52 and the plurality of second wall apertures 54. Further, upon activation, the exposure percentage of the dispenser may be from about 40% to about 90%; wherein at least about 60% of the total surface area of the plurality of apertures results from individual apertures of said plurality that each have an area of at least 4.2 mm². Specifically, the individual apertures have an area of at least about 4.2 mm².

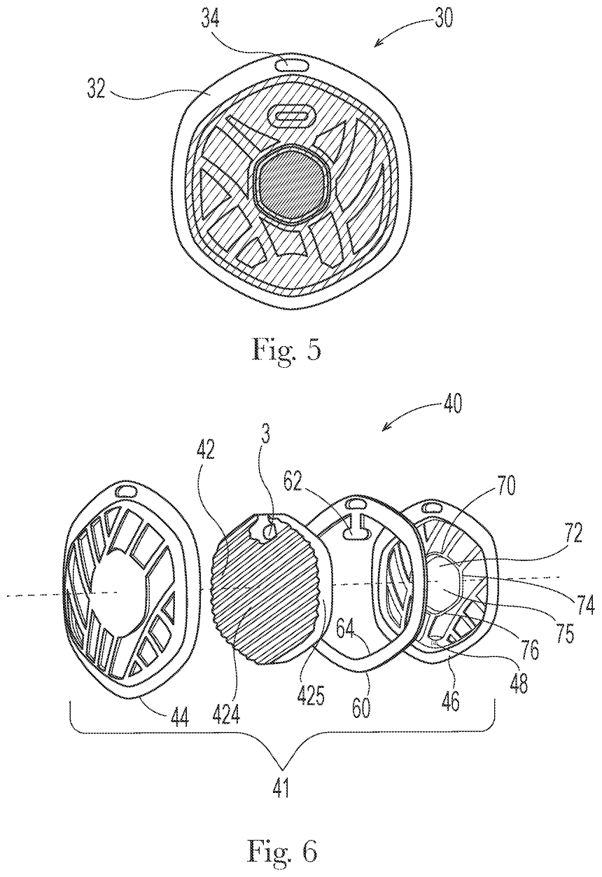

FIG. 5 is a rear view of an alternative embodiment of a volatile dispensing device 30 according to the present invention. The volatile dispensing device 30 has substantially the same components as the device 1 and differs in that the volatile dispensing device 30 comprises a flange 32 having a hanging aperture 34 for supporting the device 30 in a hanging position above a surface.

FIG. 6 is a perspective view of components in a volatile composition dispenser 40 according to the present invention. The dispenser 40 comprises a volatile dispensing device 41 and a solid article 42 comprising a volatile composition. The solid article 42 is configured to contain a solid phase of the volatile composition and comprises a plurality of evaporative surfaces to allow the solid phase of the volatile composition to evaporate therefrom. The volatile composition may be selected from the group consisting of: a perfume, a deodorizing agent, a sanitizing agent, an insect repellant, a malodor reduction agent, and mixtures thereof. The volatile composition may be present in a level of 3 wt % to 85 wt %, preferably from 15 wt % to 75 wt % of the solid article, more preferably from 20 wt % to 60 wt % of the solid article.

The volatile dispensing device 41 comprises a housing having a first wall corresponding to a front cover 44 and a second wall corresponding to a rear frame 46, which upon assembly, the front cover 44 and the rear frame 46 define a cavity for receiving the solid article 42. Each of the front cover 44 and the rear frame 46 has substantially the same features as the first wall 4 and the second wall 5 described hereinbefore and is configured for vaporizing of volatile composition in the solid article 42 into the interior space. A dispenser mount 70 extends from the rear frame 46 outwardly away from the cavity. The dispensing device 41 may further comprise an article holder 60 having an anchor 62 for releasably attaching the solid article 42 to the article holder 60. The article holder 60 may comprise an inner periphery 64 configured to define a center opening for circumscribing the solid article 42. The solid article 42 may comprise an aperture 43 sized and shaped to mate with the anchor 62. Specifically, the aperture 43 extends through a thickness of the solid article 42.

The dispenser 40 may comprise not more than one solid article, for example, the dispenser 40 may comprise a single piece of the solid article 42. Accordingly, a shape of an inner profile of the device 41 may correspond to a shape of an outer profile of the solid article 42 to minimize material use for making the device 41.

Referring to FIG. 6, the solid article 42 comprises at least one central evaporative surface 424 and a peripheral evaporative surface 425 surrounding the at least one central evaporative surface 424. The solid article 42 may further comprise a second central evaporative surface opposite the at least one central evaporative surface 424. The dispenser mount 70 may comprise a base 72 and a side wall 74 surrounding the base 72 to define a hollow center portion 75 and a dispenser rim 76. A technical effect of designing the hollow center portion 75 and the dispenser rim 76 is to minimize contact of the second central evaporative surface of the solid article 42 with the rear frame 46 so as to maximize an evaporative surface area of the solid article 42. A visual indicator 48 may be disposed on an inner surface of the rear frame 46. Specifically, the visual indicator 48 may be visible upon shrinkage of the solid article 42 from the initial size to a reduced size for providing a visual cue of a state of use of the dispenser 40.

The solid article 42 may be characterized by a shrinkage of greater than 1% to less than 40% and a technical effect is that a remaining portion of the solid article 42 has sufficient structure for the solid article 42 to be a self-supporting structure which does not require a container for supporting the solid article on a placement surface. Further, it also provides sufficient surface area for ease of removal for users to remove from the placement surface. The placement surface may be a product placement location in the interior space, such as interior surfaces, fixtures, furniture or the like.

The visual indicator 48 may be positioned anywhere in the rear frame 46 or the front cover 44 as long as it is proximal to the peripheral evaporative surface 425.

The visual indicator 48 may indicate a state of the dispenser selected from the group consisting of: an end of life of the dispenser, a quantitative state indicative of a number of days use of the dispenser, and combinations thereof. As shown in FIG. 6, the visual indicator 48 may be in the form of letters defining an English word “END” which is indicative of the end of life of the dispenser. Alternatively, the visual indicator 48 may be a set of characters or a graphical symbol indicative of the state of the dispenser.

An entire area of the central evaporative surface 424 may comprise a surface finish, such as shown in FIG. 6. A technical effect of providing a surface finish on the entire area of the central evaporative surface 424 is for ease of manufacturing, and it will be appreciated that the visual indicator 48 may also be concealed by only texturing at least a region of the central evaporative surface 424.

The volatile composition dispenser 40 may be configured in any orientation, such as in a vertical orientation during use such as shown in FIG. 7 or in a horizontal orientation as shown in FIG. 8.

FIG. 7 is a side schematic view of the assembled volatile composition dispenser 40 of FIG. 6 in a vertical orientation when the dispenser 40 is placed/attached to a first interior surface 100 or support in an interior space such as for example, a cupboard door or a wall.

Specifically, the dispenser 40 is attached to the first interior surface 100 through the dispenser mount 70. The dispenser mount 70 comprises a planar mount surface 71 that is substantially flat for supporting and/or attaching the dispenser 40 in a vertical orientation. The dispenser mount 70 is characterized by a height H configured to provide a gap 80 between the dispenser 40 and the interior surface 100 thereby preventing direct contact of the solid article 42 with the interior surface 100 and consequently minimizing risk of adverse reaction that may develop between the solid article 42 and the interior surface 100. The gap may comprise a space 80 sufficient to minimize contact and allow air flow through the apertures in the front cover 54 and the rear frame 56.

FIG. 8 is a side schematic view of the assembled volatile composition dispenser 40 of FIG. 6 in a horizontal position when the dispenser 40 is placed/attached to a second interior surface 200 or support such as for example, a table top, kitchen or bathroom countertops. The dispenser mount 70 is placed on the second interior surface 200 such that the planar mount surface 71 rests on the second interior surface 200. The dispenser mount 70 may be characterized by a height from greater than 1 mm to less than 20 mm. A technical effect is to provide a more stable support for the dispenser 40 through having a lower center of gravity in the dispenser 40.

The solid article 42 can be made of any known material that is capable of providing a self-supporting structure and having a mixture of evaporable ingredients and non-evaporable ingredients, as long as the amounts of the evaporable ingredients and non-evaporable ingredients are configured to provide shrinkage of greater than 1% to less than 40%. Evaporable ingredients may include a volatile composition and water. Non-evaporable ingredients may include ingredients suitable for forming a solid article including but not limited to gelling materials, elastomers, polyurethane or the like.

Table 1 shows an example of a water-based gel composition according to the present invention.

TABLE 1
Example of a Water-based gel composition

| | | Target (%) by weight |
|---|---|---|
| Ingredient | Function | of the composition |
| Water | Solvent | 35 |
| Carrageenan and NaCl Mixture | Gelling material | 25 |
| Gellan Gum | Gelling material | 22 |
| Calcium Chloride | Stabilizer | 15 |
| Perfume | Fragrance | 1 |
| Hydrogenated Castor Oil | Emulsifier | 2 |

The solid article may be non-aqueous, preferably comprises less than 1% by weight of water, more preferably substantially free of water.

The solid article may comprise a material selected from the group consisting of: an ethyl cellulose polymer, a chemically cross-linked polyol or derivative thereof, and mixtures thereof.

Table 2 shows an example of a non-aqueous gel composition comprising an ethyl cellulose polymer which is capable of having a shrinkage of 1% to 40%.

TABLE 2
Non-aqueous gel composition

| | Target Range (wt % by |
|---|---|
| Ingredient | weight of the composition) |
| Poly(propylene oxide), weight average | 20 to 75 |
| molecular weight of 300 to 1,000 Daltons | |
| Ethyl cellulose polymer | 0.5 to 30 |
| C₁₋₅ alkyl monoester of a C₈₋₂₂ fatty acid | 20 to 75 |
| Perfume | 4.5 to 50 |
| water | 0 to 1.5 |

The solid article may be a chemically cross-linked polyol, wherein the polyol or derivative thereof is selected from the group consisting of: polyol, polyester polyol, polyglycerol and mixtures thereof, preferably the polyol derivative is a polyester polyol, more preferably castor oil, even more preferably the polyester polyol is cross-linked using a cross-linking agent selected from the group consisting of: isocyanates, isothiocynates and mixtures thereof.

The solid article may also be molded with a moldable material such as any one of the non-aqueous gel compositions described hereinafter.

Non-Aqueous Gel Composition:

The gel composition is formed using a cross-linking agent which forms covalent bonds which are stable mechanically and thermally, so once formed are difficult to break. In contrast, physical cross-links rely on changes in the microstructure to achieve stability, such as crystalline regions or regions of high entanglement.

While physical gels can also hold high levels of hydrophobic material such as perfume, the processing of such physical gels is more delicate, as they are more readily broken during manipulation. In addition, such physical gels typically exhibit larger reductions in volume as the hydrophobic material evaporates, in comparison to the cross-linked gels of the present invention, typically at the level of from 50% to 90% by length reduction as the hydrophobic material evaporates. In contrast, the cross-linked gels of the present invention exhibit less shrinkage as the hydrophobic material is released, typically of the order of from 1% to 40%, preferably from 3% to 30%, more preferably from 4% to 20%, at the end of a time period of 1 to 75 days, preferably from 1 to 60 days, more preferably from 1 to 45 days.

The gel composition can have an elastic modulus G′ of above 0.1 kPa, preferably above 1 kPa, even more preferably above 2 kPa, and below 100 kPa.

The gel can be a chemically cross-linked polyol or derivative thereof. Suitable polyols or derivatives thereof can be selected from the group consisting of: polyol, polyester polyol, polyglycerol and mixtures thereof. Polyols, polyester polyols and polyglycerols comprise multiple hydroxyl groups, and are suitable for forming gels having a compact network. In addition, the resultant gel has greater affinity for hydrophobic materials which are less strongly hydrophobic.

Suitable polyols or derivatives thereof can have a molecular weight of from 60 Da to 10000 Da, preferably from 150 Da to 3000 Da, even more preferably from 500 Da to 2000 Da, even more preferably 600 Da to 1300 Da. Longer polyols and derivatives thereof, result in greater flexibility of the gel.

Suitable polyols and derivatives thereof do not comprise terminal hydroxyl groups. Secondary alcohols are particularly suitable. Primary alcohols, having terminal hydroxyl groups, typically result in more linear chains and a more compact network. A combination of primary and secondary alcohols are preferred, since they result in a more desired correlation length.

An average correlation length of less than 8 nm as measured using Small Angle X-Ray Scattering (SAXS) is preferred. However, the gel compositions, described herein, can be formulated to have any desired correlation length.

A gel with more optimal pore size is achieved when secondary alcohols are used. Lightly branched polyols and derivatives thereof, such as poly(diethyleneglycol adipates) result in more flexible gels. Preferred polyols and derivatives thereof have at least 2 hydroxyl groups per molecule, more preferably at least 3 hydroxyl groups per molecule.

A polyol is a compound containing multiple hydroxyl groups. Diol polyols, having two hydroxyl-functional groups, result after cross-linking in linear polymers or more open networks having large pore size. In contrast, hydroxyl-functional monomers with functionality larger than two form more compact gels with smaller pore sizes. Suitable polyols include: ethylene glycol, propylene glycol, 1,4-butanediol, 1,6-hexanediol, pentaerythritol, 1,2,6-hexanetriol, 4,6-di-tert-butylbenzene-1,2,3-triol, propanetriol (glycerol), 1,2,5-hexanetriol, 1,2,4-cyclohexanetriol, 2,5-dimethylhexane-1,2,6-triol, 3-hydroxymethylpentane-1,2,5-triol, 1,3,6-hexanetriol, 1,1,5,5-pentanetretraol, 1,2,5,6-hexanetretraol, 1,2,3,4,5,6-hexanehexol (sorbitol) and mixtures thereof. Polyester polyols are hydroxyl-containing esters. Suitable polyester polyols can be selected from the group consisting of: aliphatic polyester polyols, aromatic polyester polyols, organic oil based polyester polyols, and mixtures thereof. Organic oil based polyester polyols are preferred. Preferred organic oils are vegetable oils since they typically comprise high levels of unsaturation (C═C bonds) and naturally comprise hydroxyl groups. Suitable polyester polyols include: hexanoic acid, 4-hydroxy-, 1,1′,1″-(1,2,3-propanetriyl) ester; pentanoic acid, 5-amino-4-hydroxy-, 1,1′,1″-(1,2,3-propanetriyl) ester; Polycaprolactone triol; castor oil, hydroxyl sunflower oil (HSO) and mixtures thereof.

Castor oil is particularly suitable. Castor oil (Ricinus oil) is a pale yellow and viscous liquid, derived from the bean of the castor plant (Ricinus communis). Castor oil is predominately made up of triglycerides of fatty acids that contain 87-90% of ricinoleic acid (cis-12-hydroxyoctadec-9-enoic acid) and can be achieved in high purity grades. Castor oil and its derivatives have been used as polyols for polyurethanes and adhesives. The castor oil can be partially hydrogenated. It has been found that castor oil provides the length of the branches and the position of the hydroxyl groups which is particularly suited for providing a chemically cross-linked gel having a pore size which results in slow release of the hydrophobic material, particularly where the hydrophobic material is a perfume. In addition, the chemically cross-linked gels derived from castor oil show less syneresis of the hydrophobic material from the gel.

Polyglycerols are hydroxy-containing ethers. Polyglycerols are typically obtained by the polymerization of alkylene oxides (such as epoxides). Suitable alkylene oxides include ethylene oxide, propylene oxide, butylene oxide, and mixtures thereof, using chain initiators such as ethylene glycol, propylene glycol, diethylene glycol, dipropylene glycol, 1,4-butanediol, neopentyl glycol, pentaerythritol, hexanetriol, sorbitol, glycerol, and mixtures thereof. Suitable polyglycerols can be selected from the group consisting of: α,α-diglycerol, α,β-diglycerol, hyperbranched polyglycerol, dendritic polyglycerol, and mixtures thereof. Hyperbranched polyglycerols are aliphatic polyethers with multiple hydroxyl end groups that are obtained from the nonsymmetric polyaddition of glycidol to glycerol resulting in a globular branch-on-branch structure which provides special internal flexibility. Dendritic polyglycerols are a hyperbranched polyglycerol with a well-defined symmetric and spherical three-dimensional structure around a core. Apart from improving gel elasticity, the dendritic structure with sterically shielded core together with the exceptionally high number of functional groups of hyperbranched polyglycerols produces flexible gels with relatively low pore size, which increase the longevity of final composition by reducing the diffusion rate not only as a consequence of physically entrapping the hydrophobic material, but also enhancing H-bonding and Van der Waals interactions. Such polyglycerols can be purchased from Nanopartica GmbH (Germany) and Sigma-Aldrich. Suitable polyglycerols include: polyethylene glycol, polypropylene glycol, poly(diethylene glycol), poly(dipropylene glycol), poly(1,4-butanediol), poly(neopentyl glycol), poly(1,6-hexanediol), and mixtures thereof. The polyglycerol preferably has from 2 to 50, preferably from 4 to 30 repeat units.

Any suitable cross-linking agent can be used, though cross-linking agents selected from the group consisting of: isocyanates, isothiocynates and mixtures thereof, are preferred. The cross-linking agent can be a linear, branched, or cyclic isocyanate, and mixtures thereof. Cyclic isocyanates and mixtures thereof are preferred. Suitable cyclic isocyanates include heterocyclic isocyanates such as 1,3,5-tris(5-isocyanatopentyl)-1,3,5-triazine-2,4,6(1H,3H,5H)-trione. Suitable cross-linking agents can be selected from the group consisting of: 1, 4-butane diisocyanate (BDI), 1,6 hexamethylene diisocyanate (HMDI), L-Lysine ethyl ester diisocyanate (LDI), 4,4′-Methylenebis(cyclohexyl isocyanate) (H12MDI), Glycolide-ethylene glycol-Glycolide isocyanate (Bezwada, LLC), 4,4′-Methylenebis(phenyl isocyanate) (MDI), 2,4′-Methylenebis(phenylisocyanate) (MDI), 2,2′-Methylenebis(phenyl isocyanate) (MDI), Isophorone diisocyanate (IPDI), 2,4-toluene diisocyanate (2,4-TDI), 2,6-toluene diisocyanate (2,6-TDI), Poly (hexamethylene diisocyanate) (PDI), 1,3-bis(2-isocyanatopropan-2-yl)benzene, Poly (pentamethylene diisocyanate) and mixtures thereof, preferably 1,6 hexamethylene diisocyanate (HMDI), L-Lysine ethyl ester diisocyanate (LDI), Poly (pentamethylene diisocyanate), Poly (hexamethylene diisocyanate) (PDI), 1,3,5-tris(5-isocyanatopentyl)-1,3,5-triazine-2,4,6(1H,3H, 5H)-trione, and mixtures thereof. Such cross-linking agents are available from Sigma-Aldrich and from Covestro under trade name of Desmodur® eco N 7300.

The cross-linking agent can have a viscosity below 2.500 mPa·s at 25° C. and an isocyanate equivalent weight of from 15% to 40%, preferably from 18% to 30%. Such cross-linking agents are more easily blended with the polyol. As a result, more uniform gels can be achieved.

The gel is preferably essentially free, or free of unreacted isocyanates and/or isothiocyanates.

The gel can further comprise a hydroxyl containing polymer, a hydroxyl containing oligomer or mixtures thereof. The hydroxyl containing polymer and/or oligomer can be used to alter the elasticity of the gel composition, and therefore the longevity of the perfume release. since a higher elastic modulus G′ slows the perfume release.

Suitable hydroxyl containing polymers can be selected from the group consisting of: poloxamers, gelatins, carrageenan, chitin, chitosan, and mixtures thereof.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Suitable poloxamers have a weight average molecular weight of from 1500 g/mol to 15000 g/mol and a poly(ethylene oxide) weight percentage of from 10% to 80%, preferably from 50% to 80%. Suitable poloxamers are commercially available under the tradename of Pluronic® from BASF.

Gelatins are typically translucent, colorless, and typically obtained from collagen from various animal body parts. They are commonly used as a gelling agent in food, pharmaceutical industry, vitamin capsules, photography, and cosmetic manufacturing. Suitable gelatines can have a bloom of from 90 to 300. Bloom is a test to measure the strength of a gel or gelatin and is measured according to the method outlined by Bloom in U.S. Pat. No. 1,540,979. The test determines the weight in grams needed by a plunger with a diameter of 0.5 inch (12.7 mm) to depress the surface of the gel 4 mm without breaking it, at a temperature of 25° C. The result is expressed in Bloom (grades). It is usually between 30 and 300 Bloom. To perform the Bloom test on gelatin, a 6.67% by weight gelatin solution is kept for 17-18 hours at 10° C. prior to being tested.

Carrageenan are sulfated polysaccharide for instance derived from red algae, commonly known as Irish moss. They are typically composed principally of alpha-D-galactopyranose-4-sulfate units and 3,6-anhydro-alpha-D-galactopyranose units. At least three forms are known, designated, respectively, as “iota”, “kappa” and “lambda” carrageenan which differ in the ratios of the two galactopyranose units and accordingly in their sulfate ester content.

Kappa-carrageenan is the principal component in aqueous extracts from Chondrus crispus and Gigartina stellata. It is lower in sulfate ester content than iota and lambda carrageenan.

Chitosan is typically obtained by deacetylation under alkaline conditions of chitin, which is the second most abundant biopolymer in nature, after cellulose. Chitin can be found as an important constituent of the exoskeleton in animals, especially in crustaceans, molluscs, and insects, and it is also the principal polymer in the cell wall of certain fungi. Chitin and chitosan are linear polysaccharides composed of randomly distributed β-(1-4)-linked D-glucosamine (deacetylated unit) and N-acetyl-D-glucosamine (acetylated unit). Chitosan has two types of reactive groups that can be grafted: the free amine groups on the deacetylated units and the hydroxyl groups on the C3 and C6 carbons on acetylated or deacetylated units.

The chitosan of the present invention may have a molecular weight from 10,000 g/mol to 4,000,000 g/mol, preferably from 70,000 g/mol to 1,600,000 g/mol. Suitable chitosan may have a degree of de-acetylation of at least 50%, preferably at least 60%, more preferably at least 70%, even more preferably at least 75%.

The gel composition can be transparent or even translucent. The gel composition can have any suitable shape, such as star, circular or pyramidal. The gel composition may be colored by adding dye. The gel compositions of the present invention can be molded or even 3D printed to the desired shape.

The gel composition can be any suitable shape or size since both define the evaporative surface area of the gel composition. It is known that the shape and size of the gel composition can affect the release and longevity of the hydrophobic material. For instance, thin sheets result in faster release and lower longevity than spheres of the same mass of the gel composition. Suitable gel compositions can have an evaporative surface area of less than 150 cm2, preferably from 3.0 to 100 cm2, more preferably from 6.0 to 60 cm2.

The evaporative surface area can be measured by creating a 3D model of the gel composition using CAD software and using the CAD software to calculate the surface area. Any suitable CAD software can be used, such as AutoCAD® 2013.

The following examples are intended to more fully illustrate the present invention and are not to be construed as limitations of the present invention since many variations thereof are possible without departing from the scope of the present invention. All parts, percentages and ratios used herein are expressed as percent weight unless otherwise specified.

### EXAMPLES

Solid articles suitable for implementing in a volatile dispensing device and a volatile composition dispenser according to the present invention are prepared based on the composition details described in Table 3 below and are evaluated for the respective shrinkage (results shown in Table 4).

TABLE 3

| | | Inventive Sample | | | | | |
|---|---|---|---|---|---|---|---|
| | | (% by weight of the solid article) | | | | | |
| Component | Material | 1 | 2 | 3 | 4 | 5 | 6 |
| Polyester Polyol | Castor oil | 29 | 29 | 29 | 29 | 29 | 29 |
| Cross-linking | Isocyanate | 21 | 21 | 21 | 21 | 21 | 21 |
| Agent | | | | | | | |
| Volatile | Mixture of | 50 | 50 | 50 | 50 | 50 | 30 |
| Composition | perfume raw | | | | | | |
| | materials | | | | | | |
| Total | | 100 | 100 | 100 | 100 | 100 | 100 |

Inventive Samples of solid articles made with the composition details described in Table 1 according to the process described below.

### Process of Making:

The polyol or derivative thereof, is mixed with the volatile composition, and optionally a hydroxyl containing polymer is added. Then, the cross-linking agent is added at a temperature from 5° C. to 35° C., preferably 15° C. to 30° C. and further mixed in order to provide a homogeneous mixture. The mixture is poured into a mould of the desired shape, and cured, preferably at a curing temperature from 20° C. to 30° C. Such temperatures limit the evaporation of volatile components of the hydrophobic material. Alternatively, the mixture can be kept at 5° C. or less, in order to avoid curing. Curing will then start only once the temperature is raised to the curing temperature.

The polyol or derivative thereof can be mixed with the cross-linking agent, and optionally a hydroxyl containing polymer, preferably at a temperature from 20° C. to 85° C., more preferably from 30° C. to 75° C., for 10 min to 10 hours, preferably from 15 min to 2 hours. The mixture is cooled down and the hydrophobic material is added preferably at a temperature from 10° C. to 40° C., more preferably 15° C. to 30° C. and can be further mixed, for instance, from 15 to 120 min. The mixture is poured into the desired mould and cured, preferably at a temperature from 20° C. to 30° C. Alternatively, all of the components of the gel composition can be blended at a low temperature, such as 5° C. or less, before the temperature is increased to the curing temperature.

### Results

Shrinkage results of the Inventive Samples 1 to 6 of Table 3 are shown in Table 4 below. Specifically, the shrinkage is determined based on Formula (1) below:

Shrinkage=(Initial Length−Reduced Length)/Initial Length×100%
wherein,
Initial Length=length or diameter of the solid article before use
Reduced Length=length or diameter of the solid article after a predetermined time period of use.

TABLE 4

| | | | | Shrinkage (of | |
|---|---|---|---|---|---|
| | | | | Length/ | |
| | | Initial | | Diameter) | % |
| | | Dimensions | | at 45 Days | Shrink- |
| In- | | (first | | after use | age |
| ventive | Sample | configuration | Weight | (second | at 45 |
| Sample | Shape | before use) | (g) | configuration) | days |
| 1 | Circle | Diameter = 40 mm | 5.8 | 4.2 mm | 10.5 |
| | | Thickness = 5 mm | | | |
| 2 | Oval 1 | Length × Width × | 5.8 | 5.4 mm | 10.0 |
| | | Thickness = | | | |
| | | 54 × 26.5 × 5 mm | | | |
| 3 | Oval | Length × Width × | 8.2 | 5.6 mm | 10.4 |
| | | Thickness = | | | |
| | | 54 × 26.5 × 5 mm | | | |
| 4 | Rectangle | Length × Width = | 8.2 | 7 mm | 12.5 |
| | | 56 × 32 × 5 mm | | | |
| 5 | Rectangle | Length × Width = | 8.2 | 5 mm | 10.2 |
| | | 49 × 27 × 5 mm | | | |
| 6 | Rectangle | Length × Width = | 8.2 | 3.1 mm | 6.3 |
| | | 49 × 27 × 5 mm | | | |

All the above Inventive Samples 1 to 6 have a shrinkage of 6.3% to 12.5%, i.e. less than 40%. As a result, each of the samples at the end of life is a single unitary piece having a size sufficient for easy removal and thereby reducing mess. In contrast, conventional gels have a gelling polymer which remains after water and perfume evaporates, and the remaining gelling polymer causes stickiness in the gel. Further, the remaining gel also stick to the container break easily and causes messy residue.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A volatile dispensing device comprising:
a) a housing having opposing first and second walls that are joined along their peripheries to one another, wherein each of the first and second walls has at least one aperture and define a cavity for receiving a solid article comprising a volatile composition;
b) a dispenser mount located at a center of one of the first wall and the second wall, and extending from an exterior surface of the one of the first wall and the second wall, wherein the dispenser mount comprises a planar mount surface and is characterized by a height greater than a thickness of the one of the first wall and the second wall.

2. The device according to claim 1, wherein the dispenser mount is characterized by a height of from greater than 1 mm to less than 20 mm.

3. The device according to claim 1, wherein the planar mount surface is characterized by a mount surface area, and the one of the first wall and second wall comprises a peripheral wall region surrounding the dispenser mount, wherein the peripheral wall region is characterized by a peripheral wall region area, wherein the ratio of the mount surface area to the peripheral wall region area is from 1:2 to 1:15.

4. The device according to claim 1, wherein the dispenser mount is integral with the one of the first wall and the second wall, and wherein the dispenser mount is characterized by a thickness corresponding to a thickness of the one of the first wall and the second wall.

5. The device according to claim 1, wherein each of the first and second walls comprises a plurality of apertures, wherein, upon activation, the exposure percentage is from about 40% to about 90%; wherein at least about 60% of the total surface area of the plurality of apertures results from individual apertures of said plurality.

6. The device of claim 1, wherein the individual apertures have an area of at least about 4.2 mm².

7. The device of claim 1, wherein the first and second walls are hingedly connected to form a unitary housing piece.

8. The device of claim 1, wherein at least one of the first and second walls comprises a material selected from the group consisting of: molded pulp paper, plastic, glass, ceramic and a mixture thereof.

9. The device of claim 1, further comprising an anchor arranged within the housing for securing the solid article between the first and second walls.

10. A volatile composition dispenser comprising a device according to claim 1, and a solid article disposed in the device.

11. The dispenser according to claim 10, wherein the dispenser comprises not more than a single solid article, preferably wherein a shape of an inner profile of the device corresponds to a shape of an outer profile of the solid article.

12. The dispenser according to claim 10, wherein the solid article comprises a volatile composition selected from the group consisting of: a perfume, a deodorizing agent, a sanitizing agent, an insect repellant, a malodor reduction agent, and mixtures thereof, and wherein the volatile composition is present in a level of 3 wt % to 85 wt % of the solid article.

13. The dispenser according to claim 10, wherein the solid article is non-aqueous comprising less than 1% by weight of water.

14. The dispenser according to claim 10, wherein the solid article comprises a cellulosic substrate impregnated with and/or coated with the volatile composition.

15. The dispenser according to claim 10, wherein the solid article comprises a peripheral evaporative surface, wherein the solid article is capable of shrinkage relative to the peripheral evaporative surface from an initial size to a reduced size smaller than the initial size upon exposure of the solid article to the environment in an interior space wherein the solid article is characterized by a shrinkage in a direction away from the peripheral evaporative surface of greater than 1% to less than 40%.

16. The device according to claim 1, wherein the planar mount surface is characterized by a mount surface area, and the one of the first wall and second wall comprises a peripheral wall region surrounding the dispenser mount, wherein the peripheral wall region is characterized by a peripheral wall region area, wherein the ratio of the mount surface area to the peripheral wall region area is from 1:5 to 1:15.

17. The dispenser according to claim 10, wherein the solid article comprises a volatile composition selected from the group consisting of: a perfume, a deodorizing agent, a sanitizing agent, an insect repellant, a malodor reduction agent, and mixtures thereof, and wherein the volatile composition is present in a level of from 15 wt % to 75 wt % of the solid article.

18. The dispenser according to claim 10, wherein the solid article comprises a gel composition selected from the group consisting of: an ethyl cellulose polymer, a chemically cross-linked polyol or derivative thereof, and mixtures thereof.

19. The dispenser according to claim 18, wherein the solid article comprises a chemically cross-linked polyol, wherein the polyol or derivative thereof is selected from the group consisting of: polyol, polyester polyol, polyglycerol and mixtures thereof.
